(19) United States
(12) Patent Application Publication
(10) Pub. No.: US 2024/0182900 A1
(43) Pub. Date: Jun. 6, 2024

(54) MICRORNA BLOCKADE FOR THE TREATMENT OF DISEASE

(71) Applicant: YALE UNIVERSITY, New Haven, CT (US)

(72) Inventors: Jeffrey BENDER, Orange, CT (US); Vinod RAMGOLAM, New Haven, CT (US); Timur YAROVINSKY, Woodbridge, CT (US)

(21) Appl. No.: 17/773,478

(22) PCT Filed: Nov. 5, 2020
(86) PCT No.: PCT/US2020/059065

§ 371 Date: Apr. 29, 2022

Related U.S. Application Data
(60) Provisional application No. 62/932,205, filed on Nov. 7, 2019.

(51) Int. Cl. C12N 15/113

(52) U.S. Cl. CPC C12N 15/1136; C12N 2310/141; C12N 2310/315; C12N 2310/3231

(57) ABSTRACT

In various aspects and embodiments the invention provides compositions and methods useful in the treatment of disease. In one aspect the invention provides a composition comprising a polynucleotide comprising one or more of SEQ ID NOS: 1-41 and at least one modification selected from the group consisting of locked nucleic acid, bridged nucleic acid, phosphorothioate nucleic acid, and peptide nucleic acid.

### CROSS-REFERENCE TO RELATED APPLICATION

The present application is entitled to priority under 35 U.S.C. § 119(e) to U.S. Provisional Patent Application No. 62/932,205 filed Nov. 7, 2019, which is hereby incorporated by reference in its entirety herein.

### STATEMENT REGARDING FEDERALLY SPONSORED RESEARCH OR DEVELOPMENT

This invention was made with government support under AI124116 awarded by the National Institutes of Health. The government has certain rights in the invention.

### BACKGROUND OF THE INVENTION

MicroRNAs have emerged as a common mechanism of gene regulation that are believed to function by binding and downregulating target messenger RNA transcripts.

### SUMMARY OF THE INVENTION

In one aspect, the invention provides a composition comprising a polynucleotide comprising one or more of SEQ ID NOS: 1-41 and at least one modification selected from the group consisting of locked nucleic acid, bridged nucleic acid, phosphorothioate nucleic acid and peptide nucleic acid.

In various embodiments of the above aspect or any other aspect of the invention delineated herein, the polynucleotide is a locked nucleic acid with a phosphorothioate backbone.

In certain embodiments, the composition further comprises at least one pharmaceutically acceptable excipient.

In another aspect, the invention provides a method of treating breast cancer in a subject in need thereof, the method comprising administering to the subject a polynucleotide comprising at least 5 consecutive nucleotides from SEQ ID NO: 2, SEQ ID NO: 39, SEQ ID NO: 40 or SEQ ID NO: 41.

In certain embodiments, the polynucleotide reduces the level of expression of CD274 or VEGFA in the subject, thereby treating the breast cancer.

In another aspect, the invention provides a method of treating melanoma in a subject in need thereof, the method comprising administering to the subject a polynucleotide comprising at least 5 consecutive nucleotides from SEQ ID NO: 29, SEQ ID NO: 30, SEQ ID NO: 31 or SEQ ID NO: 32.

In certain embodiments, the polynucleotide reduces the level of expression of LIF in the subject, thereby treating the melanoma.

In another aspect, the invention provides a method of treating a colorectal cancer in a subject in need thereof, the method comprising administering to the subject a polynucleotide comprising at least 5 consecutive nucleotides from SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 17 or SEQ ID NO: 18, SEQ ID NO: 19, SEQ ID NO: 21 or SEQ ID NO: 22.

In certain embodiments, the polynucleotide reduces the level of expression of BCL2, IFNG, IL1b or IL22 in the subject, thereby treating the colorectal cancer.

In another aspect, the invention provides a method of treating cardiovascular disease in a subject in need thereof, the method comprising administering to the subject a polynucleotide comprising at least 5 consecutive nucleotides from SEQ ID NO: 27.

In certain embodiments, the polynucleotide reduces the level of expression of KLF5 in the subject, thereby treating the cardiovascular disease.

In certain embodiments, the cardiovascular disease is brain ischemia.

In certain embodiments, the cardiovascular disease is atherosclerosis.

In another aspect, the invention provides a method of treating a neurodegenerative disease in a subject in need thereof, the method comprising administering to the subject a polynucleotide comprising at least 5 consecutive nucleotides from SEQ ID NO: 23, SEQ ID NO: 24, SEQ ID NO: 25, SEQ ID NO: 26, SEQ ID NO: 33, SEQ ID NO: 34, SEQ ID NO: 35, SEQ ID NO: 36, SEQ ID NO: 37 or SEQ ID NO: 38.

In certain embodiments, the polynucleotide reduces the level of expression of TNF or IL23a in the subject, thereby treating the neurodegenerative disease.

In certain embodiments, the neurodegenerative disease is Parkinson's disease.

In certain embodiments, the neurodegenerative disease is Alzheimer's disease.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

As used herein, the term “locked nucleic acid” refers to a modified RNA nucleotide or polynucleotide with a covalent bond between the 2′ oxygen and the 4′ carbon of the pentose.

As used herein, the term “bridged nucleic acid” refers to a modified RNA nucleotide or polynucleotide with a bridging structure that limits the degrees of morphological freedom relative to unmodified nucleic acids.

As used herein, the term “phosphorothioate nucleic acid” refers to a modified RNA nucleotide or polynucleotide in which the phosphodiester bond has been replaced with a phosphorothioate bond.

As used herein, the term “peptide nucleic acid” refers to a modified RNA nucleotide or polynucleotide in which natural nucleotide bases are linked to a peptide-like backbone instead of the sugar-phosphate backbone found in DNA and RNA.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Compositions

In various aspects and embodiments, the invention provides compositions comprising an oligonucleotide complementary to a mir466l-3p target site on certain targets set forth in Table 1. Although miRNAs are typically understood to promote the degradation or block translation of their target mRNAs, this relationship is reversed with respect to mir466l-3p and certain targets such that mir466l-3p binding to the target increases, rather than decreases, the expression of the target. Therefore, without wishing to be limited by theory or bound to a particular use, the compositions of the invention are target site blockers (TSB), which bind to a mir466l-3p target site and the 5′ and 3′ flanking sequence on the target mRNA and prevent the binding of mir466l-3p. This blocks the enhancing effect of mir466l-3p on the level of expression of the target resulting in a lower level of the target.

TABLE 1
mir4661-3p Target Sites

| Gene | | | |
|---|---|---|---|
| Symbol | Gene Description | NCBI Ref | 3′-5′ Complement |
| BCL2 | B cell | NM_000633 | SEQ ID NO: 1 |
| | leukemia/lymphoma 2 | | GTCTTAAATAAATAAATCTTT |
| CD274 | Programmed Death | NM_001267706.1 | SEQ ID NO: 2 |
| (PD-L1) | Ligand 1 | | AATCAGGAATAAATATAATGCTA |
| IFNG | interferon gamma | NM_000619 | SEQ ID NO: 3 |
| | | | GTTAAATATTAATAAATAGATTTA |
| | | | SEQ ID NO: 4 |
| | | | TCCCCATATAAATAATGTTAA |
| | | | SEQ ID NO: 5 |
| | | | TGCTATTATAAATACTTATTT |
| | | | SEQ ID NO: 6 |
| | | | GAATTATAAATAATAC |
| IL1b | interleukin 1 beta | NM_000576 | SEQ ID NO: 7 |
| | | | TAAATAAATAAATAGGGA |
| | | | SEQ ID NO: 8 |
| | | | CAAATAAATAAATAAATAGG |
| | | | SEQ ID NO: 9 |
| | | | CAAACAAATAAATAAATAAAT |
| | | | SEQ ID NO: 10 |
| | | | CATTTATAAATATTCCCA |
| IL12a | interleukin 12a | NM_000882 | SEQ ID NO: 11 |
| | | | AATAGCTTATAAATATTTGCCC |
| | | | SEQ ID NO: 12 |
| | | | TATATAAATAAATATTTTAAA |
| | | | SEQ ID NO: 13 |
| | | | ATACACTTGATAAATAATTTAATA |
| | | | SEQ ID NO: 14 |
| | | | ATATAACATAAATAAATATAAGTT |
| | | | SEQ ID NO: 15 |
| | | | TATAACATAAATAAATATA |
| | | | SEQ ID NO: 16 |
| | | | TAATTTAATAAATATAACATAAA |
| IL22 | Interleukin 22 | NM_016971 | SEQ ID NO: 17 |
| | | | TTATCAATAAATATCTATGC |
| | | | SEQ ID NO: 18 |
| | | | CATTTATAAATATAAAG |
| | | | SEQ ID NO: 19 |
| | | | TAATAATAAATACATTT |
| | | | SEQ ID NO: 20 |
| | | | AATGATATAAATAAAATGC |
| | | | SEQ ID NO: 21 |
| | | | TTTCTATAAATAAATCC |
| | | | SEQ ID NO: 22 |
| | | | ATTGTCATAAATATCAAT |
| IL23A | interleukin 23 subunit | NM_016584 | SEQ ID NO: 23 |
| | alpha | | CCATCTAATAAATACTCAAC |
| | | | SEQ ID NO: 24 |
| | | | GGAGGATAAATAATCCCC |
| | | | SEQ ID NO: 25 |
| | | | AAATACAATAAATAATCCT |
| | | | SEQ ID NO: 26 |
| | | | TCAATATAAATACAATAAA |
| KLF5 | Kruppel like factor 5 | NM_001730 | SEQ ID NO: 27 |
| | | | CCACAAAATAAATAAAGATA |
| LIF | Leukemia Inhibitory | NM_001257135 | SEQ ID NO: 28 |
| | Factor | | TATCATAATAAATAGAAATC |
| | | | SEQ ID NO: 29 |
| | | | GTTAATATAAATAGGATATC |
| | | | SEQ ID NO: 30 |
| | | | TTAAATAATAAATAAGGGCC |
| | | | SEQ ID NO: 31 |
| | | | ACATTGAATAAATAAAAA |
| | | | SEQ ID NO: 32 |
| | | | AAAAATAAATATTTTAA |
| TNF | tumor necrosis factor | NM_000594.4 | SEQ ID NO: 33 |
| | | | GCAAACATAAATAGAGGGAG |
| | | | SEQ ID NO: 34 |
| | | | TAAATAATAAATAATCACA |
| | | | SEQ ID NO: 35 |
| | | | TAAATAATAAATAAATAAT |
| | | | SEQ ID NO: 36 |
| | | | CTGTAAATAAATAAATAA |
| | | | SEQ ID NO: 37 |
| | | | TCCCAAATAAATACATTC |
| | | | SEQ ID NO: 38 |
| | | | TTAATCAGATAAATATTTTAA |
| VEGFA | vascular endothelial | NM_001171629 | SEQ ID NO: 39 |
| | growth factor A | | CATCTCATAAATAGTTGAAA |
| | | | SEQ ID NO: 40 |
| | | | CACCAATAAATAAATGATAAC |
| | | | SEQ ID NO: 41 |
| | | | TAAAAATAAATATGTACT |

Accordingly, in one aspect the invention provides a composition comprising a polynucleotide comprising at least a portion of one or more of SEQ ID NOS: 1-41 and at least one modification selected from the group consisting of locked nucleic acid, bridged nucleic acid, phosphorothioate nucleic acid and peptide nucleic acid. In various embodiments the polynucleotide is a locked nucleic acid with a phosphorothioate backbone.

As discussed in more detail below, the composition may be formulated to facilitate delivery by various routes of administration. In various embodiments, the composition further comprises at least one pharmaceutically acceptable excipient. In various embodiments, the polynucleotide comprises at least 5, at least 6, at least 7, at least 8, at least 9, at least 10 consecutive nucleotides or all of one or more of SEQ ID NOS: 1-41.

### Methods of Treating Disease

In another aspect, the invention provides a method of treating disease in a subject in need thereof by providing a therapeutically effective amount of a pharmaceutical composition comprising an oligonucleotide complementary to a target site on the target mRNA mir466l-3p. In various embodiments, the target and therefore the disease are selected from the table below which contains targets known to be regulated by mir466l-3p and to play a role in various diseases. In various embodiments, the composition is administered locally to a target area.

TABLE 2
Targets

| | | | | Neuro- |
|---|---|---|---|---|
| Breast | | Colorectal | Cardiovascular | degenerative |
| Carcinoma | Melanoma | cancers | Disease | Disease |
| CD274 (PD-L1) | LIF | BCL2 | KLF5 | IL23a |
| VEGFA | | IFNG | | TNF |
| | | IL1b | | |
| | | IL22 | | |

In various aspects and embodiments, the invention provides a method of treating breast cancer in a subject in need thereof, the method comprising administering to the subject a polynucleotide comprising at least 5 consecutive nucleotides from SEQ ID NO: 2, SEQ ID NO: 39, SEQ ID NO: 40 or SEQ ID NO: 41. In various embodiments, the polynucleotide reduces the level of expression of CD274 or VEGFA in the subject, thereby treating the breast cancer.

In various aspects and embodiments, the invention provides a method of treating melanoma in a subject in need thereof, the method comprising administering to the subject a polynucleotide comprising at least 5 consecutive nucleotides from SEQ ID NO: 29, SEQ ID NO: 30, SEQ ID NO: 31 or SEQ ID NO: 32. In various embodiments, the polynucleotide reduces the level of expression of LIF in the subject, thereby treating the melanoma.

In various aspects and embodiments, the invention provides a method of treating a colorectal cancer in a subject in need thereof, the method comprising administering to the subject a polynucleotide comprising at least 5 consecutive nucleotides from SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 17 or SEQ ID NO: 18, SEQ ID NO: 19, SEQ ID NO: 21 or SEQ ID NO: 22. In various embodiments, the polynucleotide reduces the level of expression of BCL2, IFNG, IL1b or IL22 in the subject, thereby treating the colorectal cancer.

In various aspects and embodiments, the invention provides a method of treating cardiovascular disease in a subject in need thereof, the method comprising administering to the subject a polynucleotide comprising at least 5 consecutive nucleotides from SEQ ID NO: 27. In various embodiments, the polynucleotide reduces the level of expression of KLF5 in the subject, thereby treating the cardiovascular disease. In various embodiments, the cardiovascular disease is brain ischemia. In various embodiments, the cardiovascular disease is atherosclerosis.

In various aspects and embodiments, the invention provides a method of treating a neurodegenerative disease in a subject in need thereof, the method comprising administering to the subject a polynucleotide comprising at least 5 consecutive nucleotides from SEQ ID NO: 23, SEQ ID NO: 24, SEQ ID NO: 25, SEQ ID NO: 26, SEQ ID NO: 33, SEQ ID NO: 34, SEQ ID NO: 35, SEQ ID NO: 36, SEQ ID NO: 37 or SEQ ID NO: 38. In various embodiments, the neurodegenerative disease is Parkinson's Disease. In various embodiments, the polynucleotide reduces the level of expression of TNF or IL23a in the subject, thereby treating the neurodegenerative disease. In various embodiments, the neurodegenerative disease is Alzheimer's Disease.

### Administration/Dosage/Formulations

The regimen of administration may affect what constitutes an effective amount. The therapeutic formulations may be administered to the subject either prior to or after the onset of the noted inflammatory diseases. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat an inflammatory disease in the patient. An effective amount of the therapeutic compound necessary to achieve a therapeutic effect may vary according to factors such as the state of the disease or disorder in the patient; the age, sex, and weight of the patient; and the ability of the therapeutic compound to treat an inflammatory disease in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of an inflammatory disease in a patient.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of an inflammatory disease in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The present invention also includes a multi-layer tablet comprising a layer providing for the delayed release of one or more compounds of the invention, and a further layer providing for the immediate release of a medication for treatment of certain diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of an inflammatory disease in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### ENUMERATED EMBODIMENTS

Embodiment 1 provides a composition comprising a polynucleotide comprising one or more of SEQ ID NOS: 1-41 and at least one modification selected from the group consisting of locked nucleic acid, bridged nucleic acid, phosphorothioate nucleic acid and peptide nucleic acid.

Embodiment 2 provides the composition according to embodiment 1, wherein the polynucleotide is a locked nucleic acid with a phosphorothioate backbone.

Embodiment 3 provides the composition of embodiment 1, further comprising at least one pharmaceutically acceptable excipient.

Embodiment 4 provides a method of treating breast cancer in a subject in need thereof, the method comprising administering to the subject a polynucleotide comprising at least 5 consecutive nucleotides from SEQ ID NO: 2, SEQ ID NO: 39, SEQ ID NO: 40 or SEQ ID NO: 41.

Embodiment 5 provides the method of embodiment 4, wherein the polynucleotide reduces the level of expression of CD274 or VEGFA in the subject, thereby treating the breast cancer.

Embodiment 6 provides a method of treating melanoma in a subject in need thereof, the method comprising administering to the subject a polynucleotide comprising at least 5 consecutive nucleotides from SEQ ID NO: 29, SEQ ID NO: 30, SEQ ID NO: 31 or SEQ ID NO: 32.

Embodiment 7 provides the method of embodiment 6, wherein the polynucleotide reduces the level of expression of LIF in the subject, thereby treating the melanoma.

Embodiment 8 provides a method of treating a colorectal cancer in a subject in need thereof, the method comprising administering to the subject a polynucleotide comprising at least 5 consecutive nucleotides from SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 17 or SEQ ID NO: 18, SEQ ID NO: 19, SEQ ID NO: 21 or SEQ ID NO: 22.

Embodiment 9 provides the method of embodiment 8, wherein the polynucleotide reduces the level of expression of BCL2, IFNG, IL1b or IL22 in the subject, thereby treating the colorectal cancer.

Embodiment 10 provides a method of treating cardiovascular disease in a subject in need thereof, the method comprising administering to the subject a polynucleotide comprising at least 5 consecutive nucleotides from SEQ ID NO: 27.

Embodiment 11 provides the method of embodiment 10, wherein the polynucleotide reduces the level of expression of KLF5 in the subject, thereby treating the cardiovascular disease.

Embodiment 12 provides the method of embodiment 10, wherein the cardiovascular disease is brain ischemia.

Embodiment 13 provides a method of treating a neurodegenerative disease in a subject in need thereof, the method comprising administering to the subject a polynucleotide comprising at least 5 consecutive nucleotides from SEQ ID NO: 23, SEQ ID NO: 24, SEQ ID NO: 25, SEQ ID NO: 26, SEQ ID NO: 33, SEQ ID NO: 34, SEQ ID NO: 35, SEQ ID NO: 36, SEQ ID NO: 37 or SEQ ID NO: 38.

Embodiment 14 provides the method of embodiment 13, wherein the polynucleotide reduces the level of expression of TNF or IL23a in the subject, thereby treating the neurodegenerative disease.

Embodiment 15 provides the method of embodiment 13, wherein the neurodegenerative disease is Parkinson's disease.

Embodiment 16 provides the method of embodiment 13, wherein the neurodegenerative disease is Alzheimer's disease.

Embodiment 17 provides the method of embodiment 10, wherein the cardiovascular disease is atherosclerosis.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a polynucleotide comprising one or more of SEQ ID NOS: 1-41 and at least one modification selected from the group consisting of locked nucleic acid, bridged nucleic acid, phosphorothioate nucleic acid and peptide nucleic acid.

2. The composition according to claim 1, wherein the polynucleotide is a locked nucleic acid with a phosphorothioate backbone.

3. The composition of claim 1, further comprising at least one pharmaceutically acceptable excipient.

4. A method of treating breast cancer in a subject in need thereof, the method comprising administering to the subject a polynucleotide comprising at least 5 consecutive nucleotides from SEQ ID NO: 2, SEQ ID NO: 39, SEQ ID NO: 40 or SEQ ID NO: 41.

5. The method of claim 4, wherein the polynucleotide reduces the level of expression of CD274 or VEGFA in the subject, thereby treating the breast cancer.

6. A method of treating melanoma in a subject in need thereof, the method comprising administering to the subject a polynucleotide comprising at least 5 consecutive nucleotides from SEQ ID NO: 29, SEQ ID NO: 30, SEQ ID NO: 31 or SEQ ID NO: 32.

7. The method of claim 6, wherein the polynucleotide reduces the level of expression of LIF in the subject, thereby treating the melanoma.

8. A method of treating a colorectal cancer in a subject in need thereof, the method comprising administering to the subject a polynucleotide comprising at least 5 consecutive nucleotides from SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 17 or SEQ ID NO: 18, SEQ ID NO: 19, SEQ ID NO: 21 or SEQ ID NO: 22.

9. The method of claim 8, wherein the polynucleotide reduces the level of expression of BCL2, IFNG, IL1b or IL22 in the subject, thereby treating the colorectal cancer.

10. A method of treating cardiovascular disease in a subject in need thereof, the method comprising administering to the subject a polynucleotide comprising at least 5 consecutive nucleotides from SEQ ID NO: 27.

11. The method of claim 10, wherein the polynucleotide reduces the level of expression of KLF5 in the subject, thereby treating the cardiovascular disease.

12. The method of claim 10, wherein the cardiovascular disease is brain ischemia.

13. A method of treating a neurodegenerative disease in a subject in need thereof, the method comprising administering to the subject a polynucleotide comprising at least 5 consecutive nucleotides from SEQ ID NO: 23, SEQ ID NO: 24, SEQ ID NO: 25, SEQ ID NO: 26, SEQ ID NO: 33, SEQ ID NO: 34, SEQ ID NO: 35, SEQ ID NO: 36, SEQ ID NO: 37 or SEQ ID NO: 38.

14. The method of claim 13, wherein the polynucleotide reduces the level of expression of TNF or IL23a in the subject, thereby treating the neurodegenerative disease.

15. The method of claim 13, wherein the neurodegenerative disease is Parkinson's disease.

16. The method of claim 13, wherein the neurodegenerative disease is Alzheimer's disease.

17. The method of claim 10, wherein the cardiovascular disease is atherosclerosis.


SEQUENCE LISTING (41 sequences)

SEQ ID NO: 1 - DNA, 21 bp, Artificial
  Target Site Blocker (TSB) sequence
gtcttaaataaataaatcttt

SEQ ID NO: 2 - DNA, 23 bp, Artificial
  Target Site Blocker (TSB) sequence
aatcaggaataaatataatgcta

SEQ ID NO: 3 - DNA, 24 bp, Artificial
  Target Site Blocker (TSB) sequence
gttaaatattaataaatagattta

SEQ ID NO: 4 - DNA, 21 bp, Artificial
  Target Site Blocker (TSB) sequence
tccccatataaataatgttaa

SEQ ID NO: 5 - DNA, 21 bp, Artificial
  Target Site Blocker (TSB) sequence
tgctattataaatacttattt

SEQ ID NO: 6 - DNA, 16 bp, Artificial
  Target Site Blocker (TSB) sequence
gaattataaataatac

SEQ ID NO: 7 - DNA, 18 bp, Artificial
  Target Site Blocker (TSB) sequence
taaataaataaataggga

SEQ ID NO: 8 - DNA, 20 bp, Artificial
  Target Site Blocker (TSB) sequence
caaataaataaataaatagg

SEQ ID NO: 9 - DNA, 21 bp, Artificial
  Target Site Blocker (TSB) sequence
caaacaaataaataaataaat

SEQ ID NO: 10 - DNA, 18 bp, Artificial
  Target Site Blocker (TSB) sequence
catttataaatattccca

SEQ ID NO: 11 - DNA, 22 bp, Artificial
  Target Site Blocker (TSB) sequence
aatagcttataaatatttgccc

SEQ ID NO: 12 - DNA, 21 bp, Artificial
  Target Site Blocker (TSB) sequence
tatataaataaatattttaaa

SEQ ID NO: 13 - DNA, 24 bp, Artificial
  Target Site Blocker (TSB) sequence
atacacttgataaataatttaata

SEQ ID NO: 14 - DNA, 24 bp, Artificial
  Target Site Blocker (TSB) sequence
atataacataaataaatataagtt

SEQ ID NO: 15 - DNA, 19 bp, Artificial
  Target Site Blocker (TSB) sequence
tataacataaataaatata

SEQ ID NO: 16 - DNA, 23 bp, Artificial
  Target Site Blocker (TSB) sequence
taatttaataaatataacataaa

SEQ ID NO: 17 - DNA, 20 bp, Artificial
  Target Site Blocker (TSB) sequence
ttatcaataaatatctatgc

SEQ ID NO: 18 - DNA, 17 bp, Artificial
  Target Site Blocker (TSB) sequence
catttataaatataaag

SEQ ID NO: 19 - DNA, 17 bp, Artificial
  Target Site Blocker (TSB) sequence
taataataaatacattt

SEQ ID NO: 20 - DNA, 19 bp, Artificial
  Target Site Blocker (TSB) sequence
aatgatataaataaaatgc

SEQ ID NO: 21 - DNA, 17 bp, Artificial
  Target Site Blocker (TSB) sequence
tttctataaataaatcc

SEQ ID NO: 22 - DNA, 18 bp, Artificial
  Target Site Blocker (TSB) sequence
attgtcataaatatcaat

SEQ ID NO: 23 - DNA, 20 bp, Artificial
  Target Site Blocker (TSB) sequence
ccatctaataaatactcaac

SEQ ID NO: 24 - DNA, 18 bp, Artificial
  Target Site Blocker (TSB) sequence
ggaggataaataatcccc

SEQ ID NO: 25 - DNA, 19 bp, Artificial
  Target Site Blocker (TSB) sequence
aaatacaataaataatcct

SEQ ID NO: 26 - DNA, 19 bp, Artificial
  Target Site Blocker (TSB) sequence
tcaatataaatacaataaa

SEQ ID NO: 27 - DNA, 20 bp, Artificial
  Target Site Blocker (TSB) sequence
ccacaaaataaataaagata

SEQ ID NO: 28 - DNA, 20 bp, Artificial
  Target Site Blocker (TSB) sequence
tatcataataaatagaaatc

SEQ ID NO: 29 - DNA, 20 bp, Artificial
  Target Site Blocker (TSB) sequence
gttaatataaataggatatc

SEQ ID NO: 30 - DNA, 20 bp, Artificial
  Target Site Blocker (TSB) sequence
ttaaataataaataagggcc

SEQ ID NO: 31 - DNA, 18 bp, Artificial
  Target Site Blocker (TSB) sequence
acattgaataaataaaaa

SEQ ID NO: 32 - DNA, 17 bp, Artificial
  Target Site Blocker (TSB) sequence
aaaaataaatattttaa

SEQ ID NO: 33 - DNA, 20 bp, Artificial
  Target Site Blocker (TSB) sequence
gcaaacataaatagagggag

SEQ ID NO: 34 - DNA, 19 bp, Artificial
  Target Site Blocker (TSB) sequence
taaataataaataatcaca

SEQ ID NO: 35 - DNA, 19 bp, Artificial
  Target Site Blocker (TSB) sequence
taaataataaataaataat

SEQ ID NO: 36 - DNA, 18 bp, Artificial
  Target Site Blocker (TSB) sequence
ctgtaaataaataaataa

SEQ ID NO: 37 - DNA, 18 bp, Artificial
  Target Site Blocker (TSB) sequence
tcccaaataaatacattc

SEQ ID NO: 38 - DNA, 21 bp, Artificial
  Target Site Blocker (TSB) sequence
ttaatcagataaatattttaa

SEQ ID NO: 39 - DNA, 20 bp, Artificial
  Target Site Blocker (TSB) sequence
catctcataaatagttgaaa

SEQ ID NO: 40 - DNA, 21 bp, Artificial
  Target Site Blocker (TSB) sequence
caccaataaataaatgataac

SEQ ID NO: 41 - DNA, 18 bp, Artificial
  Target Site Blocker (TSB) sequence
taaaaataaatatgtact